(19) United States
(12) Patent Application Publication
(10) Pub. No.: US 2021/0260339 A1
(43) Pub. Date: Aug. 26, 2021

(54) METHODS AND SYSTEMS FOR CATHETER TARGET LOCKING

(71) Applicant: CANON U.S.A., INC., MELVILLE, NY (US)

(72) Inventor: Brian Ninni, Woburn, MA (US)

(21) Appl. No.: 17/182,910

(22) Filed: Feb. 23, 2021

Related U.S. Application Data
(60) Provisional application No. 62/980,909, filed on Feb. 24, 2020.
(60) Provisional application No. 63/132,070, filed on Dec. 30, 2020.
(60) Provisional application No. 63/132,358, filed on Dec. 30, 2020.

(51) Int. Cl. A61M 25/01; A61B 10/04; G16H 40/60; G16H 40/40

(52) U.S. Cl. CPC A61M 25/0113; A61M 25/0147; A61M 25/0127; G16H 30/20; G16H 40/60; G16H 40/40; A61M 2025/0166; A61B 10/04

(57) ABSTRACT

Methods and systems are provided for maintaining alignment of an articulated medical device which allows a reference element or reference trajectory to be set at a location within a subject. A medical tool can be guided through the device and facilitating medical procedures, including endoscopes, cameras, and biopsy tools, to a target when the articulated medical device is subjected to external forces that cause a deviation in the trajectory towards the target.

### CROSS-REFERENCE TO RELATED APPLICATIONS

The present application claims priority to U.S. provisional application No. 62/980,909 filed Feb. 24, 2020; U.S. provisional application No. 63/132,070 filed Dec. 30, 2020; and U.S. provisional application No. 63/132,358 filed Dec. 30, 2020, the disclosures of which is hereby incorporated by reference in their entirety for all purposes. Priority benefit is claimed under 35 U.S.C. § 119(e).

### FIELD OF THE DISCLOSURE

The present disclosure relates generally to apparatus and methods for medical application. More particularly, the subject disclosure is directed to an articulated medical device having a hollow cavity, wherein the device is capable of maneuvering within a patient, and allowing a medical tool to be guided through the hollow cavity for medical procedures, including endoscopes, cameras, catheters and other tools.

### BACKGROUND OF THE DISCLOSURE

Bendable medical instruments such as endoscopic surgical instruments and catheters are well known and continue to gain acceptance in the medical field. The bendable medical instrument generally includes a flexible body commonly referred to as a sleeves or sheaths. One or more tool channels extend along (typically inside) the flexible body to allow access to a target located at a distal end of the body.

The instrument is intended to provide flexible access within a patient, with at least one curve or more leading to the intended target, while retaining torsional and longitudinal rigidity so that a physician can control the tool located at the distal end of the medical instrument by maneuvering the proximal end of the medical instrument.

Recently, to enhance maneuverability of the distal end of the instrument, robotized instruments that control distal portions have emerged. In those robotized instruments, to create curves locally at the distal portion by robotics, different techniques have been disclosed.

By way of example, United States patent publication number 2016/0067450, provides multiple conduits to retain the shape of the proximal part, while the driving tendons are bending the distal part in the medical instruments. The multiple conduits would be controlled selectively in a binary way by constraining or unconstraining the proximal ends of the conduits. By selecting the constrained conduits, the bendable medical device can change the length of bending distal segment by changing the stiffness of the bendable medical device based on the area where the conduits deploy.

However, there remains a need in the industry to further refine and advance the targeting of such bendable medical devices as they can be subjected to external forces that can alter the alignment of the device tip with the target. Methods and systems for maintaining alignment with the target are therefore desired.

### SUMMARY

Thus, to address such exemplary needs in the industry, the presently disclosed system teaches method for targeting a region of interest in a subject. A medical apparatus is provided, where the medical apparatus comprises: a bendable body, at least one control wire slideably situated in the bendable body, and one or more sensor. The method next includes advancing the medical apparatus to a first location in the subject, determining a reference element, the reference element associated with the bendable body location, defining an acceptable deviation between the first location and the reference element, and countering external forces on the bendable body to maintain a location or position within the acceptable deviation.

The invention as provided herein provides a medical apparatus comprising: a bendable body; at least one control wire slideably situated in the bendable body; one or more sensor; and a controller for causing movement of the medical apparatus. The controller determines a reference element to a region of interest, determines an acceptable deviation from the reference element; and causes the medical apparatus to bend, rotate, and/or translate in order to maintain a trajectory toward the region of interest that stays within the acceptable deviation.

In various aspects, the reference element can be determined by sensors on the apparatus, and the reference element can be a reference trajectory that is a line that extends normal to the tip surface. In other aspects, reference element is an anatomical feature, and the software can internally convert than to a ‘catheter sensor based’ reference through registration.

In other aspects, the acceptable deviations can be determined based on the reference element, and forces acting upon the apparatus can be counteracted to reduce the deviation from the reference element.

In one aspect there is provided a method of maintaining alignment of the apparatus tip with a target surface. In some aspects, there is a first step of defining a reference trajectory from the apparatus tip to the target surface. In other aspects, there is a second step of defining acceptable deviation margins from the reference trajectory, and in a third step, external forces causing deviations from the reference trajectory are counteracted in order to minimize the deviation to within the acceptable deviation margins.

In another aspect, following the second step of defining acceptable deviation margins, the user can permit the apparatus to change shape to determine if the trajectory is now within the acceptable deviation margins. If the trajectory is outside of the acceptable deviation margins, a controller can return the apparatus to the desired trajectory within the acceptable deviation margins.

Maintaining alignment of the bendable device with the target when encountering external forces or when a change in the inserted or removed tool prohibits the device from returning to the desired shape can provide clinical improvements including a decrease in the length of the procedure, a decrease in the trauma to the patient and an increase in the diagnostic yield provided by the procedure.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 is a block diagram of an exemplary bendable medical device incorporating various ancillary components, according to one or more embodiment of the subject apparatus, method or system.

FIG. 2A depicts a perspective close-up view of an exemplary bendable medical device, according to one or more embodiment of the subject apparatus, method or system.

FIG. 2B depicts a perspective view of an exemplary bendable medical device, according to one or more embodiment of the subject apparatus, method or system.

FIG. 3 provides a cut-away view of an exemplary bendable medical device inserted into a cavity, according to one or more embodiment of the subject apparatus, method or system.

FIG. 4A illustrates the defining of a reference element and an acceptable margin of deviation.

FIG. 4B illustrates a minor deviation in the trajectory that falls within the acceptable margins and would be allowed.

FIG. 4C illustrates the counteraction of a large deviation to stay within the acceptable margins.

FIG. 5A illustrates the defining of a reference element and an acceptable margin of deviation.

FIG. 5B illustrates the relaxing of the bendable body to allow insertion of a tool.

FIG. 5C illustrates that the bendable body having the tool inserted cannot bend to the original shape to match the reference element, and is instead translated in space to re-align with the trajectory.

FIG. 6A illustrates the defining of a reference element and an acceptable deviation where there are two targets.

FIG. 6B illustrates the defining of a reference element and an acceptable deviation where there are two targets.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

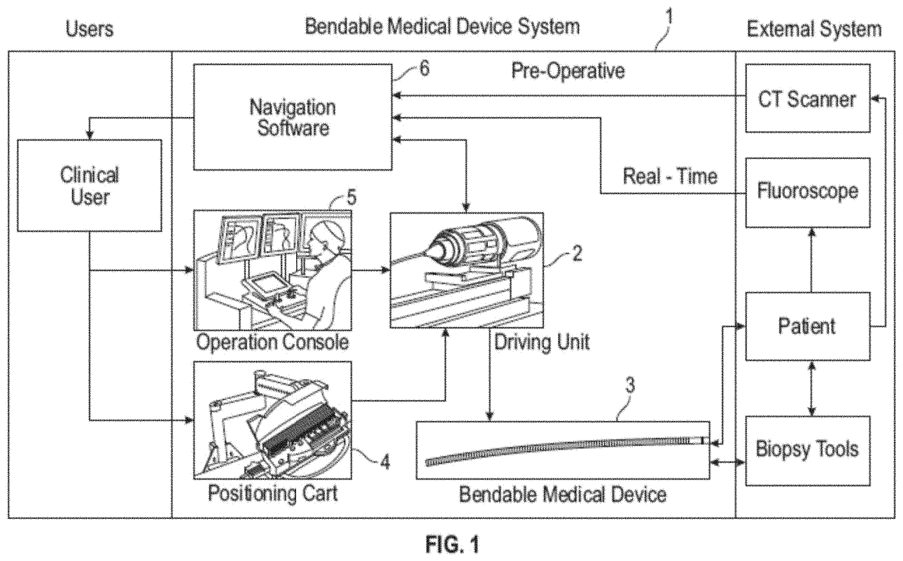

FIG. 1-3 describe the structure and functionality of exemplary bendable medical devices that can be used with the systems and methods described herein to minimize deviation from a reference element that is set from the tip of the bendable medical device towards the target. In addition to the description herein, additional descriptions of systems that can be used with the systems and methods described herein can be found in, for example, International Patent Publications WO/2018/204202; WO/2020/086749; WO/2020/092097; WO/2020/092096; and WO/2020/243285 and U.S. Pat. Publications 2019/0105468; and US 2020/0375682, each of which are incorporated herein in their entirety.

FIG. 1 is a system block diagram of an exemplary bendable medical device system 1 incorporating various ancillary components intended to amass a complete medical system. The bendable or articulated medical device system 1 comprises a driving unit 2, a bendable body 3, a positioning cart 4, an operation console 5 and navigation software 6. The exemplary bendable medical device system 1 is capable of interacting with external system component and clinical users to facilitate use in a patient.

The navigation software 6 and the driving unit 2 are communicatively-coupled via a bus to transmit/receive data between each other. Moreover, the navigation software 6 is connected and may communicate with a CT scanner, a fluoroscope and an image server (not in Figure), which are ancillary components of the bendable medical device system 1. The image server may include, but is not limited to, a DICOM™ server connected to a medical imaging device including but not limited to a CT and/or MRI scanner and a fluoroscope. The navigation software 6 processes data provided by the driving unit 2 and data provided by images stored on the image server, and/or images from the CT scanner and the fluoroscope in order to display images onto the image display.

The images from the CT scanner may be pre-operatively provided to navigation software 6. With navigation software, a clinical user creates an anatomical computer model from the images. In this particular embodiment, the anatomy is that of a lung with associated airways. From the chest images of the CT scanner, the clinical user can segment the lung airways for clinical treatments, such as biopsy. After generating the lung airway map, the user can also create plan to access the lesion for the biopsy. The plan includes the airways to insert and maneuver the bendable medical device 3 leading to the intended target, which in this example is a lesion.

The driving unit 2 comprises actuators and a control circuitry. The control circuitry is communicatively-coupled with operation console 5. The driving unit 2 is connected to the bendable medical device 3 so that the actuators in the driving unit 2 operate the bendable medical device 3. Therefore, a clinical user can control the bendable medical device 3 via the driving unit 2. The driving unit 2 is also physically connected to a positioning cart 4. The positioning cart 4 includes a positioning arm, and locates the driving unit 2 and the bendable medical device 3 in the intended position with respect to the target/patient. The clinical user can insert, maneuver and retreat the bendable medical device 3 to perform medical procedures, here a biopsy in the lungs of the patient.

The bendable medical device 3 can be navigated to the lesion in the airways based on the plan by the clinical user's operation. The bendable medical device 3 includes a hollow cavity for various tools (e.g. a biopsy tool). The bendable medical device 3 can guide the tool to the lesion of the patient. In one example, the clinical user can take a biopsy sample from the lesion with a biopsy tool.

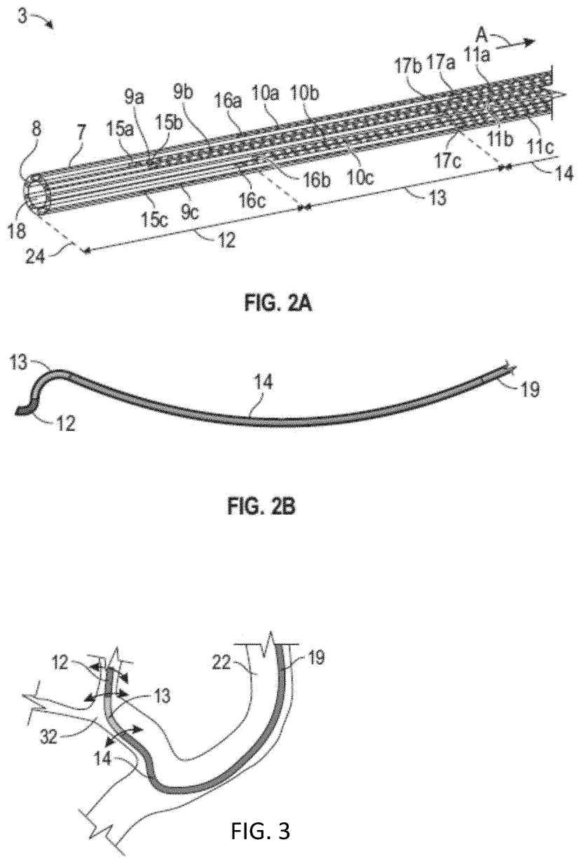

FIGS. 2A and 2B are schematic drawings of one embodiment of the bendable medical device 3. FIG. 2A is a perspective close-up view of the bendable medical device 3. FIG. 2B is a schematic drawing to explain the bendable segments of the bendable medical device 3. The bendable medical device 3 has a distal end 24 and a proximal end (in direction of arrow A), and comprises a proximal part 19 and three bendable segments, which are the first, second, and third bendable segments 12, 13, 14, respectively.

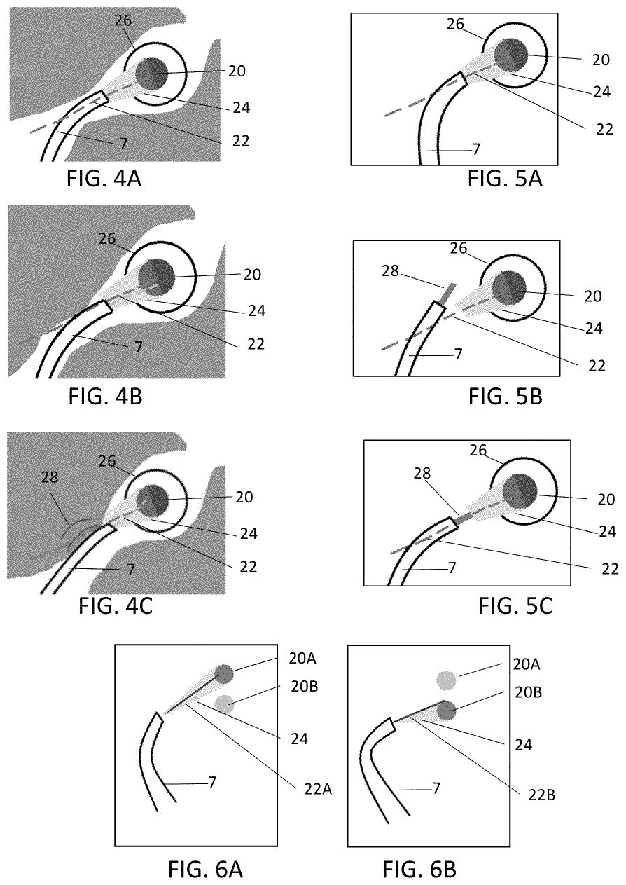

As shown in the embodiment of FIG. 2B, the bendable segments 12, 13, 14, can independently bend and can form a shape with three independent curvatures. The bendable medical device 3 includes a bendable body 7 with an inner diameter 40 and an outer diameter 42 (see FIG. 4B), which creates the cylindrical wall 8 of the bendable body 7, wherein the inner diameter establishes a hollow cavity extending the length of the bendable body that can be used as a tool channel 18 (see FIG. 4b). The wall 8 may extend the entire length of the bendable body 7 or may have portioned removed or be sectioned (e.g., having guide rings) to increase bendability. The wall 8 may house several lumens 34 intended to house a control wire. The tool channel 18 is configured to extend the length of the bendable body 7, wherein the proximal part 19 of the bendable body 7 provides access to clinical users for inserting/retreating a medical tool. For example, a clinical user can insert and retrieve a biopsy tool trough the tool channel 18 to the distal end 24 of the bendable medical device 3. This may be accomplished after the bendable device 3 is inserted into the patient, or in unison with insertion/retreating the bendable device 3.

The bendable body 7 includes a set of first control wires 9a, 9b, 9c, a second set of control wires 10a, 10b, 10c, and a third set of control wires 11a, 11b, 11c. The wall 8 houses the control wires 9a-11c in the lumens 34, which are configured along longitudinal direction of the bendable body 7. The lumens 34 allow for slideable movement of the control wires 9a-11c along an axial direction of the bendable body. The control wires 9a-11c are terminated at the distal end of each bendable segment 12, 13 and 14, to form the three bendable groups, each with three wires each (a, b, c). The first control wires 9a, 9b, 9c are terminated at the distal end of the first bendable segment 12 with anchoring segments 15a, 15b, 15c, and are configured apart from each other by approximately 120 degrees within the wall 8. The first control wires 9a, 9b, 9c are connected to the driving unit 2 at the proximal end of the wires 9a, 9b, and 9c. The driving unit 2 induces pushing or pulling forces to move the control wires 9a, 9b, and 9c by actuating those wires, and bending the bendable body 7 from the distal end 24.

Similarly, the second set of control wires 10a, 10b, 10c are terminated at the distal end of the second bendable segment 13, using the anchoring segments 16a, 16b, 16c, and are connected to the driving unit 2 at the proximal end. The second set of control wires 10a, 10b, 10c are also housed in the wall 8. The second set of control wires boa, hob, and hoc can bend the bendable body 7 from the distal end of the second bendable segment 13.

In the same way, the third set of control wires 11a, 11b, 11c are also configured to bend the bendable body 7 at the third bendable segment 14, once again by inducing pushing or pulling, actuated at the distal end 24 of the control wires 11a, 11b, 11c by the driving unit 2.

Accordingly, by pushing and pulling the set of control wires 9, 10, 11, the first, the second and the third bendable segments 12, 13, 14, respectively, individually bend the bendable medical device 3, in three dimensions.

Moreover, the bendable medical device 3 can have a continuous smooth surface on the outer diameter 42 and the inner diameter 40 of the bendable body 7 to avoid risk of trauma to the patient anatomy and improved tool advancement/retraction in the tool channel 18. Furthermore, the control wire 9, 10, 11, can be fixed to the bendable body at a wide variety of positions along the length of the bendable body 7, allowing the bendable medical device 3 to be configured to have multiple bending segments, especially a distal bending segment manipulated independently from the proximal part of the bendable body to provide improved flexible access to the intended treatment area of the patient. While a three-section bendable robot is described in this embodiment. It is contemplated that the bendable robot may have 2, 4, 5, 6 or more bendable sections, depending on the intended use. In order to counter the external forces on the bendable body to maintain a location or position, two are more bendable sections within the bendable body are preferred. This allows for using forces in, for example, the second and third bendable segments (segments 13 and 14) to adjust the pose of the medical device. The bendable body 7 may further include a sheath surrounding the bendable body. The sheath can provide a smooth outer surface and will be of a biocompatible material.

FIG. 3 provides a cut-away view of an exemplary bendable medical device 3 inserted into a cavity. FIG. 3 exemplifies the navigation and targeting of a lesion in peri-bronchial area of a patient's lungs, which is a lateral area surrounding the airways. This area is a known challenge to target as identified in literature and the prior art, due to the limited distal dexterity of the conventional catheter. To reach the lesion through airways 22 in the navigation stage, the first and the second bendable segments 12, 13, respectively, navigate the bendable medical device 3 through the bifurcation point 32. The first bendable segment 12 can adjust the shape/orientation to the daughter branch while the second bendable segment 13 can adjust the shape/orientation to the parent branch in the bifurcation point 32, as the bendable medical device 3 advances through the bifurcation point 32. Once the first and the second bendable segments 12 and 13 pass the bifurcation point 32, those segments may act as guides for the rest of the bendable medical device 3, so that the insertion force from the proximal end of the single catheter can be effectively transformed into the insertion force for a distal part of the single catheter without serious prolapsing of the distal section. Once the distal end 24 of the bendable medical device 3 reaches the vicinity of the lesion, the bendable medical device 3 would direct the distal end 24 to the lesion 23, which locates the lateral area around the airway, by bending the first and the second bendable segments 12 and 13, respectively. Since the airway doesn't directly connect with the lesion 23, this is one of the more difficult configurations for a conventional catheter.

With the first, the second and the third bendable segments 12, 13 and 14, respectively, the bendable medical device 3 can orient the distal end 24 without moving the proximal part 19 that goes through all bifurcations to this lesion. By using the three-dimensional bending capability of the first and the second bendable segments 12 and 13, the bendable medical device 3 can perform unique maneuvers to enhance capability of the peri-bronchial targeting such as omnidirectional viewing and cluster viewing, such as described in WO/2020/092097; WO/2020/086749 and US Pat. Pub. 2018/0311006)

The bendable body 7 can function as bending objects differently along the axial direction, because the control wires 9, 10, 11, are mapped to the different position of the bendable body 7. Therefore, the bendable medical device 3 can provide improved access to the intended lesion through tortuous pathways. Also, the bendable medical device 3 can have different flexibility along the axial direction without increasing the size or number of the jointing points.

Also, the third bendable segment can deform by external forces to follow the shape of tortuous pathways in the anatomy, such as lung airways, blood vessels and brain ventricles, while minimizing exerted force to the anatomy. Therefore, by following the shape of the anatomy, the third bendable segment can be navigated by the first and the second bendable sections when the catheter is inserted, and develop the delivery line for both the medial instruments for medical treatments and the driving force for control.

The deformation, or alternatively, translation, of the bendable body 7 by external forces can occur based on the shape of pathways in the anatomy, changes in the anatomy positions due to functions such as breathing, by the insertion of a tool into tool channel 18, or the movement or use of such a tool. Such deformation or translation can result in the distal end 24 being off misaligned with the target.

FIG. 4A-4C provide a method to maintain alignment of the distal end 24 with the target surface in the presence of external forces acting upon the bendable body 7. Thus, in a first step, the user can direct the distal end 24 of the bendable body 7 towards the target and set, for example, the current tip position or orientation as a reference element. Said reference element can be determined via one or more sensors on or embedded in the medical apparatus. Alternatively, the reference element may be an anatomical feature. This anatomical feature is registered to the position of the bendable body using the one or more sensors. The sensors may be on the bendable body 7, either at or near the tip or proximal from the tip, or on the control wire 9. In other embodiments, one or more sensor may be in the driving unit 2 (e.g., clamped to an actuator). The one or more sensors used for determining the trajectory may be electromagnetic (EM) sensor(s), force sensor(s), Fiber Bragg Grating(s), etc. The reference element could also be an anatomical feature, the software can internally convert than to a ‘catheter sensor based’ reference through registration.

The reference element can be a reference trajectory that is a line that is normal to the tip. In one aspect, the trajectory can be centered with respect to the tool channel 18.

The reference element may be defined with the goal of making sure the bendable body can aim at a region of interest. Thus, the reference element may be described in relation to the region of interest. In some embodiments, the reference element may be defined as the distal end of the bendable body as the bendable body is positioned sufficiently near the region of interest. In other embodiments, the reference element may be a ghost element as described in U.S. Pat. Appl. Ser. No. 63/132,070, herein incorporated by reference. Thus, the reference element may be displayed such that any movement of the bendable body away from the position from which the reference element was defined can be seen by the addition of a ghost image of the reference element.

FIG. 4A shows a bendable body 7 inserted in a lumen and facing a region of interest 20. A reference trajectory 22 is drawn from the distal tip of the bendable body 7 to a center portion of the region of interest 20 (e.g., a tumor or other lesion or a region within a tumor or lesion 26). Around the region of interest 20, an acceptable deviation 24 is determined. This acceptable deviation 24 in this embodiment corresponds to a cone extending from the proximal tip of the bendable body 7 to the outer edges of the region of interest 20 such that propagation of a tool within this cone will provide access to the region of interest 20. This margin of deviation 24 may, for example, encompass the tumor 26 or fall within a portion of the tumor 26.

In a second step, the navigation software 6 defines acceptable margins of deviation 24 from the reference element 22 in one or more directions. In one aspect, the margins defining the area of acceptable deviation are determined by setting an angle value for an error cone extending from the tip 24. In another aspect (not shown), the margin can be defined by virtually selecting a point in space relative to the tip, and setting the size of the zone that the trajectory must stay within. In a further aspect, the margin can be defined by “painting” the surface of a segmented lesion to represent the area in which the trajectory must stay within.

The acceptable deviation may be input by the clinical user by, for example, indicating the positions on a screen. In some embodiments, the acceptable deviation is derived from input from the clinical user. For example, the information from the user is combined with the position of the proximal end of the bendable body and/or a previously defined location for a region of interest. In another example, the acceptable deviation is the percent coverage or the surface area (mm²) of the segmented lesion In yet other embodiments, the acceptable deviation is pre-defined by the system based, for example, a pre-define angle value for the error cone. Alternative pre-defined acceptable deviations are calculated based on the reference element and the size of the region of interest. It may be the same size as the lesion that is segmented or smaller than this lesion, depending on the needs of the user. The acceptable deviation can be the margin of error or margin of deviation that the user finds acceptable for the targeting procedure.

In yet other embodiments, there are two (or more) acceptable deviations, a primary acceptable deviation and a broader secondary acceptable deviation, where, when the positions are within the second acceptable deviation but outside of the primary acceptable deviation, notice of this is presented to the clinical user, such as via a yellow flag.

FIG. 4B shows a situation where the actual trajectory of the bendable body 7 has a minor deviation from the reference element 22. However, this deviation is not enough such that the trajectory of the bendable body 7 or a tool extending therefrom would move outside the area of acceptable deviation 24 (also describe as acceptable margin). In contrast, FIG. 4C shows a situation where the actual trajectory of the bendable body 7 has a large deviation from the reference element 22. This can occur when the anatomy moves due to the breathing motion. Such a situation may also occur, for example, when a stiff-tipped tool is placed within a hollow cavity in the bendable body 7. In such instances, the external forces causing the discrepancy between the reference element and the current trajectory are countered.

In a third step, the external forces are counteracted to reduce the deviation from the reference element. If the forces are caused by, for example, a tool, (i.e. to motion from the application of a biopsy probe or due to the increased stiffness of the tool end) the counteraction can partially or fully resist any deviation from the desired trajectory. When the forces are caused by the anatomy, the clinical user and/or the navigation software will preferably limit the range of movement, as necessary to ensure patient safety. Force limiting can also be used to ensure integrity of the bendable medical device. In some embodiments, the external forces are countered by adjusting the control wires only for the middle bendable segment 13 to adjust the pose of the medical device to maintain a location within the acceptable margin of deviation. In other embodiments the external forces are countered by adjusting the control wires for both the middle segment 13 and the proximal segment 14.

In some embodiments, the degree of counteraction is inversely proportional to the distance from the edge of the acceptable margin. For instance, more deviation can be permitted the closer the trajectory is to the desired trajectory. As any anatomical motion may be cyclical (e.g., breathing), the clinical user and/or the navigation software can measure the impulse (integral of force over time) and stop the counteraction when a specific, defined limit is exceeded. To further ensure patient safety, motors can be back driven afterwards to ensure that a minimum of force is exerted. Further, some deviation can be permitted to reduce the impulse allowing the bendable body 7 to maintain an acceptable trajectory for a longer time span.

As the bendable medical device 3 can be controlled at multiple bending segments, the counteraction does not have to respond directly to the direction of the external force. Rather, multiple bending segments of the bendable device 3 can be adjusted as long as the final trajectory stays within the acceptable margins, as shown in FIG. 5A-C.

In the embodiment shown if FIG. 5A, the reference element 22 and acceptable margin 24 are defined. When a tool is inserted into a hollow cavity within the bendable body 7, the bend in the bendable body 7 may relax, as shown if FIG. 5B. The bendable body 7 may be deliberately relaxed in order to allow the tool 28 to pass through to the distal end of the bendable body. Alternatively, the higher stiffness of a rigid tip of a inserted tool 28 may causes the bendable body 7 to straighten compared to FIG. 5A. This means that the proximal tip of the bendable body 7 and tool 28 are no longer within the area of acceptable deviation 24, and any biopsy or other procedure will not reach the region of interest 20. FIG. 5C shows an adjustment where the external forces caused by the tool 28 are countered. While the bendable body 7 shown in FIG. 5C cannot bend to the original shape with the increased stiffness, it can translate in space to re-align the bendable body 7 sufficiently with the reference element 22 such that the direction of the bendable body 7 is within the area of acceptable deviation 24.

Specifically, since some tools have a rigid tip for the functional element, e.g. a needle and forceps, the distal bendable segment 12 would be constrained with the rigid tip and become difficult to bend. In this case, when the system readjusts the targeting orientation, movement of middle bendable segment 13, which has offset from the location of the rigid tip of the tool, can be prioritized for the adjustment.

In some embodiments, the reference element may be defined with the goal of assuring that the bendable body can aim or access multiple targets within a region of interest, as described in U.S. Pat. Appl. Ser. No. 63/132,374, herein incorporated by reference. For multiple targets (e.g., multiple biopsy locations) the reference element may be a reference trajectory to the geometric center around the various target locations. Alternatively, multiple reference elements may be defined for each of the various targets.

The user can create multiple reference elements and cycle between them. These reference elements can each be defined manually, or can be defined automatically. The user can also manually cycle between them, or have the software automatically move to the next one. Once the reference element has changed, the controller will automatically realign the bendable body with the new reference element, and maintain the pose to stay within the de-predefined acceptable deviation by countering external forces on the bendable body. Each reference element can have its own pre-defined acceptable deviation, or a single set of parameters can be applied to all references. In FIG. 6A, the bendable body 7 is originally align to the top target 20A (dark gray). Then, when the user or the system switches to the bottom target 20B as shown in FIG. 6B, the bendable body 7 adjusts to be aligned to the lower target 20B, within the acceptable deviation 24. In the exemplary embodiment of FIG. 6B, since the tops alignment of the reference element 22B when it is within the acceptable deviation 24, it will stop at the very edge of the acceptable deviation 24. This functionality can be adjusted, for example, the bendable body 7 can adjust to be in the center of the acceptable deviation 24 when the reference element is switched from 20A to 20B, and only stay within the whole bounds of the acceptable deviation 24 during the tool exchange.

In some exemplary embodiments, in a first step, the user can direct the distal end 24 of the bendable body 7 towards the target and set the current tip position (e.g., a first location) or orientation as a reference element in step one. In the second step, the User can define, have the navigation software 6 define, or modify a predefined value for acceptable deviations from the reference element in one or more directions.

In a third step the user can permit the bendable body 7 to change shape from that which the bendable body 7 was in at the time the reference element was defined. For instance, the bendable body 7 can relax a curvature or otherwise deform in order to allow for the insertion or removal of a tool, or to conform to anatomical motion (e.g., breathing). In a fourth step, the bendable body 7 is then returned to the desired trajectory within the acceptable margins.

If returning the bendable body 7 to the original shape (for instance, by using the same inputs) does not return the bendable body 7 to the desired trajectory (due to changes in stiffness due to a tool insertion or removal, or due to anatomical motion), the clinical user and/or the navigation software can use the trajectory information and the acceptable margins of deviation to minimize the deviation from the desired trajectory. For instance, the position of the bendable body 7 can be adjusted at any of the bendable segments, the bendable body 7 can be rotated such that the trajectory is rotated, the bendable body 7 can be translated in space, or any combination of these positioning changes can be utilized.

Using feedback from the sensors, the clinical user and/or the navigation software can fine-tune the orientation and position of the trajectory to fall within the acceptable margin. Such fine-tuning can be, for example, from trial and error, or from inverse-kinematics to determine the new inputs necessary.

Once the external forces on the bendable body are countered to maintain the location or position within the acceptable deviation, further adjustments to maintain the location may not be necessary. The countering step may be, for example, shortening or extending one or more control wires and, once moved, the position of the bendable body will be maintained. It is possible for the user then to, for example, take a biopsy sample with high confidence that the sample is taken from the correct location. However, in some embodiments, a continuous or repeated countering step may be preferred. Thus, multiple countering (e.g., multiple movement of one or more control wires) may be preferred.

### Software Related Disclosure

Embodiment(s) of the present disclosure can be realized by computer system 400 or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer system may comprise one or more processors (e.g., central processing unit (CPU) 410, micro processing unit (MPU)) and may include a network of separate computers or separate processors to read out and execute the computer executable instructions. The computer-executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless).

### Other Embodiments, Modifications, and/or Advantages

The various embodiments disclosed herein describe systems, methods, and computer-readable media of providing endoscope navigation guidance for controlling a bendable body such as a catheter having a proximal section attachable to an actuator and a distal section insertable into a lumen of a patient. The bendable body can be advantageously controlled to be inserted through a lumen and maintained in a positional relation with respect to a target site. The bendable body can operate with different configurations without removing it. For example, the bendable body may be inserted with an endoscope imaging device (the endoscopic camera) or other tool to view the lesion and then, once correctly positioned and the reference element determined, the imaging device can be removed and a biopsy tool can be inserted. Additional biopsy tools or other tools may also be inserted after the first biopsy is taken.

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. For example, the present disclosure has been described above in terms of exemplary embodiments. However, there are many variations not specifically described to which the present disclosure could be applicable. For example, while the various embodiments are described with respect to an endoscope for use in medical procedures, the disclosure would be also applicable with respect to mechanical procedures of a borescope for use within various mechanical structures. Therefore, the scope of the following claims is to be accorded the broadest interpretation so as to encompass all such modifications and equivalent structures and functions.

1. A medical apparatus comprising:
a bendable body;
at least one control wire slideably situated in the bendable body;
one or more sensor; and
a controller for causing movement of the medical apparatus;
wherein the controller:
determines a reference element to a region of interest;
determines an acceptable deviation from the reference element; and,
causes the medical apparatus to bend, rotate, and/or translate in order to maintain a trajectory toward the region of interest that stays within the acceptable deviation.

2. The medical apparatus of claim 1, wherein the reference element is a referent trajectory from the bendable body to the region of interest.

3. The medical apparatus of claim 1, wherein the one or more sensors is an electromagnetic sensor that determines the position of the medical device and the reference element.

4. The medical apparatus of claim 1, wherein the bendable body has a hollow cavity extending the length of the bendable body, and a wall at least partially formed about the hollow cavity.

5. The medical apparatus of claim 1, wherein the acceptable deviation is determined by one or more of:
setting an angle value for an error cone extending from a distal end of the bendable body;
selecting a point in space relative to the distal end, and setting the size of the zone that the reference element must stay within; and
selecting an area of the surface of the region of interest in which the reference element must stay within.

6. The medical apparatus of claim 1, wherein countering the external forces on the bendable body comprises causing the medical apparatus to bend such that the reference element stays within or returns to the acceptable deviation.

7. The medical apparatus of claim 1, wherein the bendable body has at least a first, second, and third bendable section from distal end to proximal, and the countering the external forces on the bendable body comprises causing at least the second bendable section to bend.

8. The medical apparatus of claim 1, wherein the controller uses feedback from the one or more sensors as to the force applied to the medical apparatus is used in conjunction with feedback on the position of the medical apparatus to maintain the reference element within the acceptable deviation.

9. A method for targeting a region of interest in a subject, comprising:
providing a medical apparatus comprising:
a bendable body;
at least one control wire slideably situated in the bendable body; and
one or more sensor;
advancing the medical apparatus to a first location in the subject;
determining a reference element, the reference element associated with the bendable body location;
defining an acceptable deviation between the first location and the reference element; and
countering external forces on the bendable body to maintain a location or position within the acceptable deviation.

10. The method of claim 9, wherein the reference element is a reference trajectory from the bendable body to the region of interest.

11. The methods of claim 9, wherein the one or more sensors is an electromagnetic sensor that determines the position of the medical device and the reference element.

12. The method of claim 9, wherein the bendable body has a hollow cavity extending the length of the bendable body, and a wall at least partially formed about the hollow cavity.

13. The method of claim 9, further comprising defining at least a second reference element and defining an acceptable deviation between the first location and the at least a second reference element.

14. The method of claim 9, wherein the acceptable deviation is determined by one or more of:
setting an angle value for an error cone extending from a distal end of the bendable body;
selecting a point in space relative to the distal end, and setting the size of the zone that the reference element must stay within; and
selecting an area of the surface of the region of interest in which the reference element must stay within.

15. The method of claim 9, wherein countering the external forces on the bendable body comprises causing the medical apparatus to bend such that the reference element stays within or returns to the acceptable deviation.

16. The method of claim 9, wherein the bendable body has at least a first, second, and third bendable section from distal end to proximal, and the countering the external forces on the bendable body comprises causing at least the second bendable section to bend.

17. The method of claim 9, wherein countering the external forces on the medical apparatus comprises one or more of rotating and translating the medical apparatus such that the reference element stays within or returns to the acceptable margins of deviation.

18. The method of claim 9, wherein feedback from the one or more sensors as to the force applied to the medical apparatus is used in conjunction with feedback on the position of the medical apparatus to maintain the reference element within the acceptable deviation.

19. The method of claim 9, wherein advancing the medical apparatus to a first location in the subject is an advancement based on directions from a manipulation unit controlled by a user.

20. A method of treating a subject comprising:
providing a medical apparatus comprising:
a bendable body;
at least one control wire slideably situated in the bendable body; and
one or more sensor;
advancing the medical apparatus to a first location in the subject;
determining a reference element, the reference element associated with the bendable body location;
defining an acceptable deviation between the first location and the reference element;
countering external forces on the bendable body to maintain a location or position within the acceptable deviation;
inserting a treating tool into the medical apparatus to a location within the acceptable deviation from the reference element; and
treating the subject.

21. The method of claim 20, wherein treating the subject comprises taking a biopsy from the subject.

22. The method of claim 21, wherein the reference element is a referent trajectory from the bendable body to an area from which a biopsy is to be obtained.
